(19) United States
(12) Patent Application Publication
(10) Pub. No.: US 2019/0062307 A1
(43) Pub. Date: Feb. 28, 2019

(54) DEUTERIUM-SUBSTITUTED QUINOLINE DERIVATIVES

(71) Applicant: CHIA TAI TIANQING PHARMACEUTICAL GROUP CO., LTD., Lianyungang City (CN)

(72) Inventors: Yinsheng ZHANG, Nanjing (CN); Yong GAO, Nanjing (CN)

(21) Appl. No.: 16/108,800

(22) Filed: Aug. 22, 2018

(30) Foreign Application Priority Data
Aug. 24, 2017 (CN) 201710734482.7

(51) Int. Cl. C07D 401/12; C07B 59/00; G01N 33/96

(52) U.S. Cl. CPC C07D 401/12; C07B 2200/05; G01N 33/96; C07B 59/002

(57) ABSTRACT

The present application provides deuterium-substituted quinoline derivatives, preparation methods thereof and uses thereof. In particular, the present application provides deuterium-substituted compounds of anlotinib, a tyrosine kinase inhibitor, and their preparation methods.

### CROSS-REFERENCE TO RELATED APPLICATIONS

This application is based on and claims priority to Chinese Patent Application No. 201710734482.7, filed on Aug. 24, 2017, the content of which is hereby incorporated by reference in its entirety.

### TECHNICAL FIELD

The present invention belongs to the field of medical chemistry, and provides herein deuterium-substituted quinoline derivatives, a preparation method thereof and use thereof.

### BACKGROUND OF THE INVENTION

Receptor tyrosine kinases are a type of enzyme that span the cell membrane, with an extracellular binding region that binds to growth factors, a transmembrane domain, and an intracellular portion. The function of the intracellular portion is to act as a kinase to phosphorylate specific tyrosine residues in proteins and affect cell proliferation. Tyrosine kinases can be divided into growth factor receptors (e.g. EGFR, PDGFR, FGFR, and erbB2) or non-receptor kinases (e.g. c-src and bcr-abl). These kinases are abnormally expressed in human cancers and are associated with a variety of cancers.

WO2008112407 discloses 1-[[[4-(4-fluoro-2-methyl-1H-indol-5-yl)oxy-6-methoxyquinolin-7-yl]]oxy]methyl]cyclopropylamine of Formula A, which can be used as a tyrosine kinase inhibitor, and which is also known as anlotinib. In view of the importance of tyrosine kinases in physiological processes, there is a need to further develop other tyrosine kinase inhibitors, including derivatives or analogues of anlotinib.

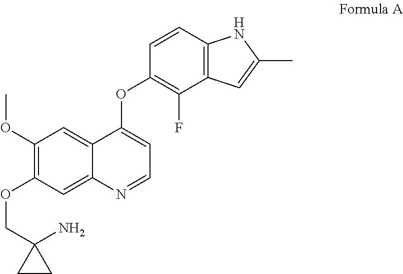 [SMILES: COc1cc2c(Oc3ccc4[nH]c(C)cc4c3F)ccnc2cc1OCC1(N)CC1]

### SUMMARY OF THE INVENTION

Accordingly, one object of the present invention is to provide deuterium-substituted quinoline derivatives or a pharmaceutically acceptable salt thereof.

It is another object of the present invention to provide a pharmaceutical composition comprising at least one of the deuterium-substituted quinoline derivatives or a pharmaceutically acceptable salt thereof.

It is another object of the present invention to provide a method for identifying the content of a quinoline analog, comprising using at least one of the deuterium-substituted quinoline derivatives or a pharmaceutically acceptable salt thereof as an internal standard.

It is another object of the present invention to provide a method of treating diseases mediated by at least one tyrosine kinase comprising administering to a mammal in need of such treatment a therapeutically effective amount of at least one of the deuterium-substituted quinoline derivatives of the present invention or a pharmaceutically acceptable salt thereof, or a pharmaceutical composition thereof.

It is another object of the present invention to provide a process for preparing the deuterium-substituted quinoline derivatives of the present invention or a pharmaceutically acceptable salt thereof.

### DETAILED DESCRIPTION

Before describing several exemplary embodiments of the invention, it is to be understood that the invention is not limited to the details of construction or process steps set forth in the following description. The invention encompasses other embodiments and can be practiced or carried out in various ways.

In a first aspect, the application provides a compound of Formula (I), or a pharmaceutically acceptable salt thereof,

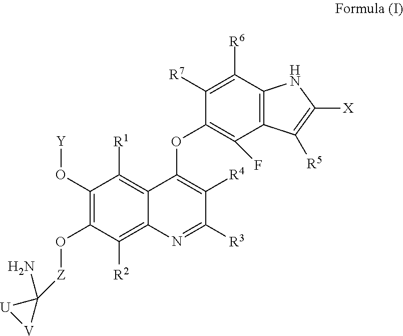

Wherein each of X, and Y is independently C(R⁸)₃; each of Z, U, and V is independently C(R⁹)₂; and each of R¹, R², R³, R⁴, R⁵, R⁶, R⁷, R⁸, and R⁹ is independently selected from hydrogen or deuterium; provided that at least one of R¹, R², R³, R⁴, R⁵, R⁶, R⁷, R⁸, and R⁹ is deuterium.

In some embodiments, at least one of R⁵, R⁶, R⁷, and R⁸ is deuterium.

In some typical embodiments, at least one of R⁵, R⁶, and R⁷is deuterium, and X is CD₃.

In some more typical embodiments, R⁵, R⁶, R⁷ are deuterium, and X is CD₃.

In some typical embodiments, Y is CD₃.

In some embodiments, at least one of R¹, R², R³, and R⁴ is deuterium.

In some typical embodiments, R³ is deuterium.

In some more typical embodiments, R³ is deuterium, and R², and R⁴ are hydrogen.

In some embodiments, at least one of R⁸, and R⁹ is deuterium.

In some typical embodiments, at least one of Z, U, and V is CD₂.

In some more typical embodiments, Z is CD₂.

In some more typical embodiments, Z is CD₂, and U and V are CH₂.

In some more typical embodiments, Z is CH₂, U and V are CD₂.

In some more typical embodiments, Z, U, and V are CD₂.

In a compound of this application, when a particular position is designated as having deuterium, it is understood that the abundance of deuterium at that position is substantially greater than the natural abundance of deuterium, which is about 0.015%.

In other embodiments, a compound of this application has an abundance of deuterium of at least 1%, at least 5%, at least 10%, at least 20%, at least 50%, at least 70%, at least 80%, at least 90%, at least 95%, at least 97%, at least 98%, or at least 99% at each designated position.

In some embodiments of the present application, the abundance of deuterium in R³,

R⁵ through R⁷, and X is at least 10%, at least 20%, or at least 30%.

In some embodiments of the present application, the abundance of deuterium in R³ is at least 50%; in some embodiments, the abundance of deuterium in R³ is at least 70%.

In some embodiments of the present application, the abundance of deuterium in R⁵ is at least 20%; in some embodiments, the abundance of deuterium in R⁵ is at least 40%; in some embodiments, the abundance of deuterium in R⁵ is at least 50%.

In some embodiments of the present application, the abundance of deuterium in R⁶ is at least 10%; in some embodiments, the abundance of deuterium in R⁶ is at least 20%; in some embodiments, the abundance of deuterium in R⁶ is at least 30%.

In some embodiments of the present application, the abundance of deuterium in R⁷ is at least 60%; in some embodiments, the abundance of deuterium in R⁷ is at least 80%; in some embodiments, the abundance of deuterium in R⁷ is at least 95%.

In some embodiments of the present application, the abundance of deuterium in X is at least 60%; in some embodiments, the abundance of deuterium in X is at least 80%; in some embodiments, the abundance of deuterium in X is at least 95%.

In some embodiments, the application provides the following exemplary embodiments:

| Cmpd | | | | | | | | | | | | |
|---|---|---|---|---|---|---|---|---|---|---|---|---|
| Num- | | | | | | | | | | | | |
| ber | R¹ | R² | R³ | R⁴ | R⁵ | R⁶ | R⁷ | X | Y | Z | U | V |
| 1 | H | H | H | H | D | D | D | CD₃ | CH₃ | CH₂ | CH₂ | CH₂ |
| 2 | H | H | H | H | D | D | D | CD₂H | CH₃ | CH₂ | CH₂ | CH₂ |
| 3 | H | H | H | H | D | D | D | CDH₂ | CH₃ | CH₂ | CH₂ | CH₂ |
| 4 | H | H | H | H | D | D | D | CH₃ | CH₃ | CH₂ | CH₂ | CH₂ |
| 5 | H | H | H | H | D | D | D | CH₃ | CH₃ | CD₂ | CH₂ | CH₂ |
| 6 | H | H | H | H | D | D | D | CH₃ | CH₃ | CH₂ | CD₂ | CD₂ |
| 7 | H | H | H | H | D | D | D | CH₃ | CH₃ | CD₂ | CD₂ | CD₂ |
| 8 | H | H | H | H | D | D | D | CH₃ | CH₃ | CH₂ | CDH | CDH |
| 9 | H | H | H | H | D | D | D | CH₃ | CH₃ | CDH | CDH | CDH |
| 10 | H | H | H | H | D | D | D | CD₃ | CH₃ | CD₂ | CH₂ | CH₂ |
| 11 | H | H | D | H | D | D | D | CD₃ | CH₃ | CH₂ | CH₂ | CH₂ |
| 12 | D | D | D | D | D | D | D | CH₃ | CH₃ | CH₂ | CH₂ | CH₂ |
| 13 | D | D | D | D | D | D | D | CD₃ | CD₃ | CH₂ | CH₂ | CH₂ |
| 14 | D | D | D | D | D | D | D | CH₃ | CH₃ | CH₂ | CD₂ | CD₂ |
| 15 | D | D | D | D | D | D | D | CH₃ | CH₃ | CD₂ | CH₂ | CH₂ |
| 16 | D | D | D | D | D | D | D | CH₃ | CH₃ | CD₂ | CD₂ | CD₂ |
| 17 | D | D | D | D | D | D | D | CD₃ | CH₃ | CD₂ | CH₂ | CH₂ |
| 18 | D | D | D | D | H | H | H | CH₃ | CH₃ | CH₂ | CH₂ | CH₂ |
| 19 | D | D | D | D | H | H | H | CD₃ | CH₃ | CH₂ | CH₂ | CH₂ |
| 20 | D | D | D | D | H | H | H | CH₃ | CD₃ | CH₂ | CH₂ | CH₂ |
| 21 | D | D | D | D | H | H | H | CH₃ | CH₃ | CD₂ | CH₂ | CH₂ |
| 22 | D | D | D | D | H | H | H | CH₃ | CH₃ | CH₂ | CD₂ | CD₂ |
| 23 | D | D | D | D | H | H | H | CH₃ | CH₃ | CD₂ | CD₂ | CD₂ |
| 24 | D | D | D | D | H | H | H | CH₃ | CH₃ | CH₂ | CDH | CDH |
| 25 | H | H | D | H | H | H | H | CH₃ | CH₃ | CH₂ | CH₂ | CH₂ |
| 26 | H | H | H | H | H | H | H | CD₃ | CD₃ | CH₂ | CH₂ | CH₂ |
| 27 | H | H | H | H | H | H | H | CH₃ | CD₃ | CH₂ | CH₂ | CH₂ |
| 28 | H | H | H | H | H | H | H | CH₃ | CD₂H | CH₂ | CH₂ | CH₂ |
| 29 | H | H | H | H | H | H | H | CH₃ | CDH₂ | CH₂ | CH₂ | CH₂ |
| 30 | H | H | H | H | H | H | H | CH₃ | CH₃ | CD₂ | CH₂ | CH₂ |
| 31 | H | H | H | H | H | H | H | CH₃ | CH₃ | CDH | CH₂ | CH₂ |
| 32 | H | H | H | H | H | H | H | CH₃ | CH₃ | CH₂ | CD₂ | CH₂ |
| 33 | H | H | H | H | H | H | H | CH₃ | CH₃ | CH₂ | CDH | CH₂ |
| 34 | H | H | H | H | H | H | H | CH₃ | CH₃ | CH₂ | CH₂ | CD₂ |
| 35 | H | H | H | H | H | H | H | CH₃ | CH₃ | CH₂ | CH₂ | CDH |
| 36 | H | H | H | H | H | H | H | CH₃ | CH₃ | CD₂ | CD₂ | CD₂ |
| 37 | H | H | H | H | H | H | H | CH₃ | CH₃ | CH₂ | CD₂ | CD₂ |
| 38 | H | H | H | H | H | H | H | CH₃ | CH₃ | CDH | CDH | CDH |
| 39 | D | D | H | D | D | D | D | CD₃ | CD₃ | CD₂ | CD₂ | CD₂ |
| 40 | D | D | D | D | D | D | D | CD₃ | CD₃ | CD₂ | CD₂ | CD₂ |

In some typical embodiments, the present application provides a compound selected from the group consisting of:

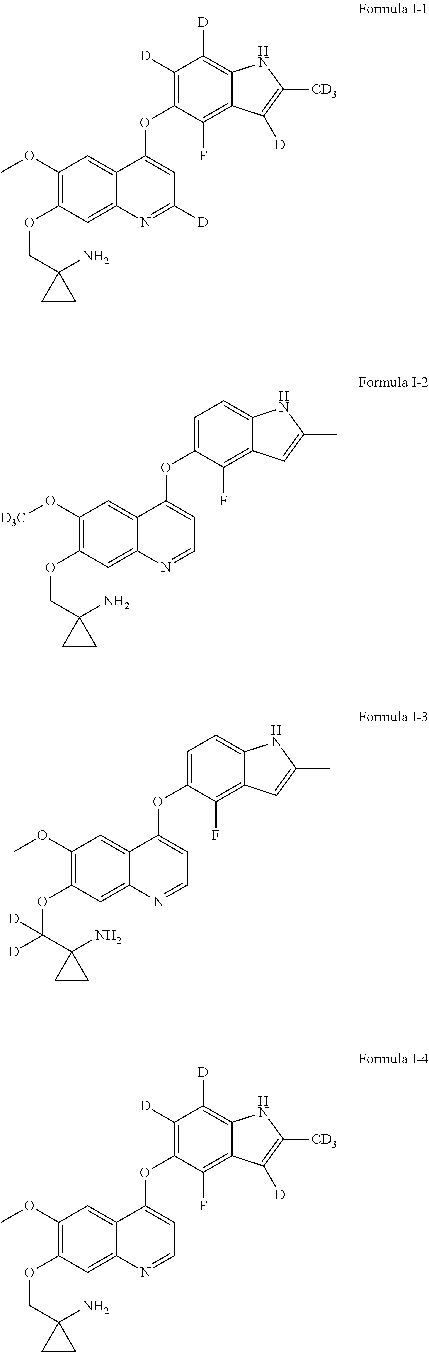

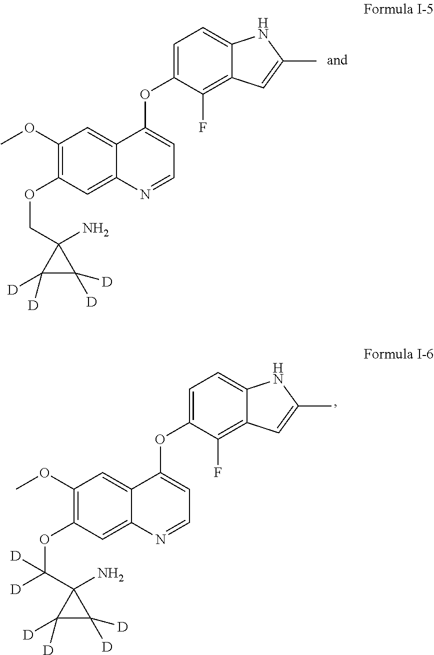

or pharmaceutically acceptable salts thereof.

In another aspect, the present application relates to a pharmaceutical composition comprising a compound of Formula (I) disclosed herein, or a pharmaceutically acceptable salt thereof. In some embodiments, the pharmaceutical composition of the present application further comprises a pharmaceutically acceptable adjuvant.

In another aspect, the present application relates to a method for determining the concentration of a quinoline analog in a sample, comprising using at least one of the deuterium-substituted quinoline derivatives of the present application or a pharmaceutically acceptable salt thereof as an internal standard. In some embodiments, the present application provides a method for determining the concentration of anlotinib or a salt thereof, comprising using at least one of the deuterium-substituted quinoline derivatives of the present application or a pharmaceutically acceptable salt thereof as an internal standard.

In another aspect, the present application relates to the use of the compound of Formula (I) disclosed herein or the pharmaceutically acceptable salt thereof as an internal standard for the analysis of 1-[[[4-(4-fluoro-2-methyl-1H-indol-5-yl)-oxy-6-methoxyquinoline-7-yl]oxy]methyl]cyclopropylamine.

In some embodiments, the present application provides a method for determining the concentration of anlotinib or the salts thereof in a sample, e.g., mammalian extracellular fluid (such as plasma and cerebrospinal fluid), comprising (1) using the compound of Formula (I) disclosed herein as an internal standard to the sample to be tested, (2) analyzing the mixture including the sample and the internal standard by a chromatographic method, and (3) determining the concentration of anlotinib.

In another aspect, the present application relates to a method of treating a disease mediated by a tyrosine kinase comprising administering to a mammal in need of such treatment, preferably a human, a therapeutically effective amount of the compound of Formula (I) disclosed herein or the pharmaceutically acceptable salt thereof, or the pharmaceutical composition thereof.

In another aspect, the present application relates to the use of the compound of Formula (I), or the pharmaceutically acceptable salt thereof, or the pharmaceutical composition thereof, for the manufacture of a medicament for preventing or treating a tyrosine kinase mediated disease.

The compound of Formula (I) may be administered in the form of its free base, or it may be administered in the form of its salts, hydrates, solvates, or prodrugs which can be converted in vivo to the free base form of the compound of Formula (I). For example, the compound of Formula (I) is administered as the pharmaceutically acceptable salt thereof. Salts can be prepared from different organic and inorganic acids by methods well known in the art within the scope of the present invention.

In some embodiments, the compound of Formula (I) is administered in the form of a hydrochloride salt. In some embodiments, the compound of Formula (I) is administered in the form of a monohydrochloride salt. In some embodiments, the compound of Formula (I) is administered in the form of a dihydrochloride salt. In some embodiments, a crystalline form of the hydrochloride salt of the compound of Formula (I) is administered. In a particular embodiment, a crystalline form of the dihydrochloride salt of the compound of Formula (I) is administered.

The compound of Formula (I), or the pharmaceutically acceptable salt thereof, can be administered by a variety of routes including, but not limited to, oral, parenteral, intraperitoneal, intravenous, intraarterial, transdermal, sublingual, intramuscular, rectal, buccal, intranasal, by inhalation, vaginal, intraocular, topical, subcutaneous, intra- and intra-articular, intraperitoneal, and intrathecal. In a particular embodiment, the compound of Formula (I) is administered orally.

The compound of Formula (I) or the pharmaceutically acceptable salt thereof can be administered one or more times a day. Preferably, a therapeutically effective amount of the compound of Formula (I), or the pharmaceutically acceptable salt thereof, is administered once daily. It may be administered in a single dose or in multiple doses, preferably in a single dose once a day. Administration of the above dosage levels of the compound of Formula (I), or the pharmaceutically acceptable salt thereof, once daily, increases patient compliance. In one embodiment, it is administered once per day, and may optionally be administered once per day in a single dose. In one embodiment, a single dose of an oral capsule is administered once per day. In all of the administration methods of the compound of Formula (I) described herein, the daily dose is from 0.01 to 200 mg/kg body weight, either alone or in divided doses.

The compound of Formula (I) or the pharmaceutically acceptable salt thereof, when administered, can maintain efficacy without administration daily, i.e., the compound of Formula (I), or the pharmaceutically acceptable salt thereof, is administered to a patient at intervals to provide a therapeutically effective amount of the compound of Formula (I) in plasma.

The interval administration includes an administration period and a withdrawal period, and the compound of the Formula (I) or the pharmaceutically acceptable salt thereof may be administered one or more times a day during the administration period. For example, the compound of the Formula (I) or the pharmaceutically acceptable salt thereof is administered daily during an administration period, and then the administration is stopped during a subsequent withdrawal period, followed by a second administration period, and then a second withdrawal period, and thus repeated. The ratio of the administration period to the withdrawal period in days is 2:0.5 to 5, preferably 2:0.5 to 3, more preferably 2:0.5 to 2, still more preferably 2:0.5 to 1.

In some embodiments, the continuous administration lasts for 2 weeks and withdrawal for 2 weeks. In some embodiments, once-daily administration lasts for 14 days, followed by withdrawal for 14 days; such dosing regimen can be repeated for a specified period of time.

In some embodiments, the continuous administration lasts for 2 weeks and withdrawal for 1 week. In some embodiments, the drug is administered once a day for 14 days, followed by 7 days of withdrawal; such dosing regimen can be repeated for a specified period of time.

In some embodiments, the continuous administration lasts for 5 days and withdrawal for 2 days. In some embodiments, the drug is administered once a day for 5 days, followed by 2 days of withdrawal; such dosing regimen can be repeated for a specified period of time.

In some embodiments, administration of the compound of Formula (I) or the pharmaceutically acceptable salt thereof at intervals as described above can not only maintain the plasma concentration of the compound in the patient below 100 ng/ml, but also achieve the therapeutic effect for a variety of tumors. The dosing regimen disclosed herein can control the drug accumulation in the patient.

In some embodiments, the compound of Formula (I), or the pharmaceutically acceptable salt thereof, is provided as a sole active ingredient in the treatment of disease mediated by a tyrosine kinase. In some embodiments, the compound of Formula (I), or the pharmaceutically acceptable salt thereof, and other anti-tumor agents are provided as active ingredients for the treatment of disease mediated by a tyrosine kinase. In some embodiments, other anti-tumor drugs include, but are not limited to, one or more of platinum complexes, fluoropyrimidine derivatives, camptothecin and its derivatives, terpenoid anti-tumor antibiotics, taxanes, mitomycin, and trastuzumab. In some embodiments, the platinum complexes include, but are not limited to, one or more of cisplatin, carboplatin, nedaplatin, and oxaliplatin; in some embodiments, the fluoropyrimidine derivatives include, but are not limited to, one or more of piraceta, fluorouracil, difurfuryluracil, deoxyfluorouridine, tegafur, and carmofur; in some embodiments, camptothecin and its derivatives include, but are not limited to, one or more of camptothecin, hydroxycamptothecin, irinotecan, and topotecan; in some embodiments, terpene anti-tumor antibiotics include, but are not limited to, one or more of doxorubicin, epirubicin, daunorubicin, and mitoxantrone; in some embodiments, the taxanes include, but are not limited to, paclitaxel, and/or docetaxel.

In another aspect, the present application relates to a process for the preparation of the compound of Formula (I), and the specific steps and routes are as follows:
(1) a compound of Formula C and a compound of Formula B are reacted in the presence of a base and a solvent to give a compound of Formula D;
(2) deprotection of the compound of Formula D to give a compound of Formula E;
(3) the compound of Formula E and a compound of Formula F are reacted in a solvent in the presence of a base to give a compound of Formula G; and
(4) the compound of Formula G is converted to a compound of Formula H;

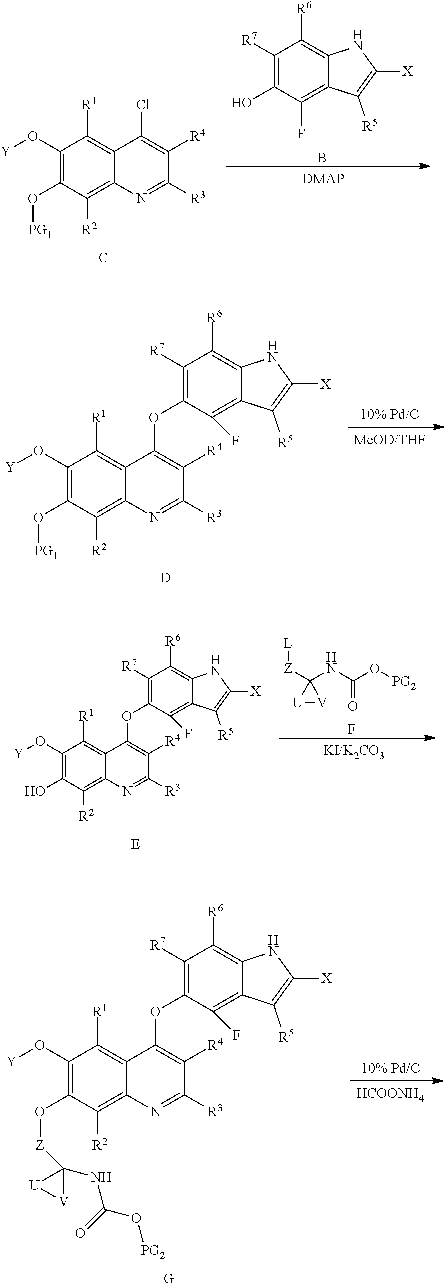

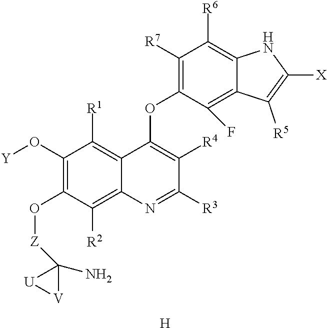

Wherein each of X, and Y is independently C(R⁸)₃; each of Z, U, and V is independently C(R⁹)₂; and each of R¹, R², R³, R⁴, R⁵, R⁶, R⁷, R⁸, and R⁹ is independently selected from hydrogen or deuterium; provided that at least one of R¹, R², R³, R⁴, R⁵, R⁶, R⁷, R⁸, and R⁹ is deuterium; PG₁ and PG₂ are protecting groups, which can be independently selected from appropriate protecting groups disclosed in the fifth edition of Greene's protective groups in organic synthesis; in some embodiments, PG₁ and PG₂ are independently benzyl, 2,4-dimethylbenzyl, 4-methoxybenzyl, 2,6-dichlorobenzyl, 3,4-dichlorobenzyl, and 4-(dimethylamino)carbonylbenzyl; L is a leaving group; in some embodiments, L is selected from OMs, OTf, OTs, and Cl.

In some embodiments, the base of step (1) is selected from the group consisting of triethylamine, diisopropylethylamine, potassium carbonate, cesium carbonate, DMAP, sodium t-butoxide, potassium t-butoxide, and sodium hydride. In some embodiments, the base of step (1) is preferably potassium carbonate or DMAP, and further preferably DMAP.

In some embodiments, the solvent described in step (1) is selected from the group consisting of 2,6-lutidine, pyridine, 1,4-dioxane, chloroform, dichloromethane, and a mixture thereof. In some embodiments, the base of step (1) is preferably 2,6-lutidine.

In some embodiments, the molar ratio of the compound of Formula B, the compound of Formula C and the base described in step (1) is 1˜1.5:1:1˜6, preferably 1˜1.2:1:1.5˜3, and further preferably 1:1:2.

In some embodiments, the reaction temperature in step (1) ranges from 100° C. to 180° C., preferably from 140° C. to 160° C., and further preferably 140° C.

In some embodiments, the compound of Formula D is subjected to a reduction reaction in a solvent to remove the protecting group under a catalyst to obtain the compound of the Formula E in step (2).

In some embodiments, the catalyst of step (2) is selected from the group consisting of 5% Pt/C, 10% Pd/C, 20% Pd/C or 50% Pd/C. In some embodiments, the catalyst of step (2) is preferably 10% Pd/C or 20% Pd/C, and further preferably 10% Pd/C.

In some embodiments, the solvent in step (2) is selected from a mixture of MeOD or MeOH and THF, a mixture of MeOD or MeOH and ethyl acetate, or a mixture of EtOD or EtOH and THF. In some embodiments, the solvent in step (2) is preferably a mixture of MeOD and THF or a mixture of MeOH and THF.

In some embodiments, the mass ratio of the compound of Formula D to the catalyst in step (2) is 1˜10:1, preferably 4˜6:1, and further preferably 4.5:1.

In some embodiments, the base of step (3) is selected from the group consisting of potassium iodide/potassium carbonate, sodium iodide/sodium carbonate, potassium iodide/sodium carbonate, and potassium iodide/cesium carbonate. In some embodiments, the base of step (3) is preferably potassium iodide/potassium carbonate, or potassium iodide/cesium carbonate, and further preferably potassium iodide/potassium carbonate.

In some embodiments, the solvent in step (3) is selected from the group consisting of 2-butanone, acetone, DMF, and a mixture thereof. In some embodiments, the solvent in step (3) is preferably 2-butanone.

In some embodiments, the molar ratio of the compound of Formula E to the compound of Formula F in step (3) is 0.5˜3:1, preferably 0.5˜1:1, and further preferably 1:1.

In some embodiments, the reaction temperature in step (3) ranges from 40° C. to 100° C., preferably 50° C. to 70° C., and further preferably 60° C.

In some embodiments, the compound of Formula G is subjected to a reduction reaction in a solvent to remove the protecting group in the presence of a catalyst and a hydrogen source to obtain the compound of Formula H in step (4).

In some embodiments, the catalyst of step (4) is selected from the group consisting of 10% Pd/C, 20% Pd/C, 50% Pd/C, and 5% Pt/C. In some embodiments, the catalyst of step (4) is preferably 10% Pd/C or 20% Pd./C, and further preferably 10% Pd/C.

In some embodiments, the hydrogen source described in step (4) is selected from the group consisting of hydrogen, hydrazine hydrate, and ammonium formate. In some embodiments, the hydrogen source described in step (4) is preferably hydrazine hydrate or ammonium formate, and further preferably ammonium formate.

In some embodiments, the solvent in step (4) is selected from the group consisting of MeOD, EtOD, and a mixture thereof. In some embodiments, the solvent in step (4) is preferably MeOD.

In some embodiments, the mass ratio of the compound of Formula G and catalyst in step (4) is 1˜10:1, preferably 1˜5:1, and further preferably 2˜2.5:1.

The molar ratio of the compound of Formula G to the hydrogen source in step (4) is 1:1˜10, preferably 1:5˜8, and more preferably 1:5.5.

In some embodiments, the reaction temperature in step (4) ranges from 25° C. to 60° C., preferably 40° C. to 60° C., and further preferably 50° C.

In some embodiments, the deuterium substituted compound of Formula B can be synthesized according to the method described below. In some embodiments, the reaction is performed in the presence of a catalyst, D₂O, and H₂ or NaBH₄. In some embodiments, the illustrative examples of catalyst include, but are not limited to platinum oxide, platinum, and palladium (such as Pd/C, palladium hydroxide, palladium oxide, palladium acetate, palladium chloride). In some embodiments, the catalyst is 10% Pd/C, PtO₂, or 5% Pt/C.

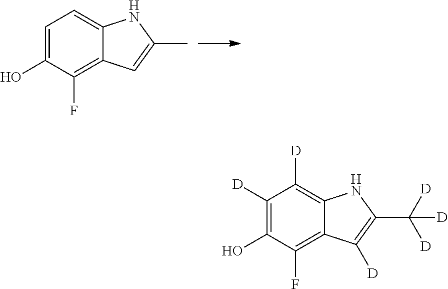 [SMILES: Cc1cc2c(F)c(O)ccc2[nH]1 ; [2H]c1c(O)c(F)c2c([2H])c(C([2H])([2H])[2H])[nH]c2c1[2H]]

In some specific embodiments, the compound of Formula I-1 or Formula I-4 can be prepared by the following route, wherein if deuterated methanol is used in the last step, then the compound of Formula I-1 is obtained; if methanol is used, then the compound of Formula I-4 is obtained.

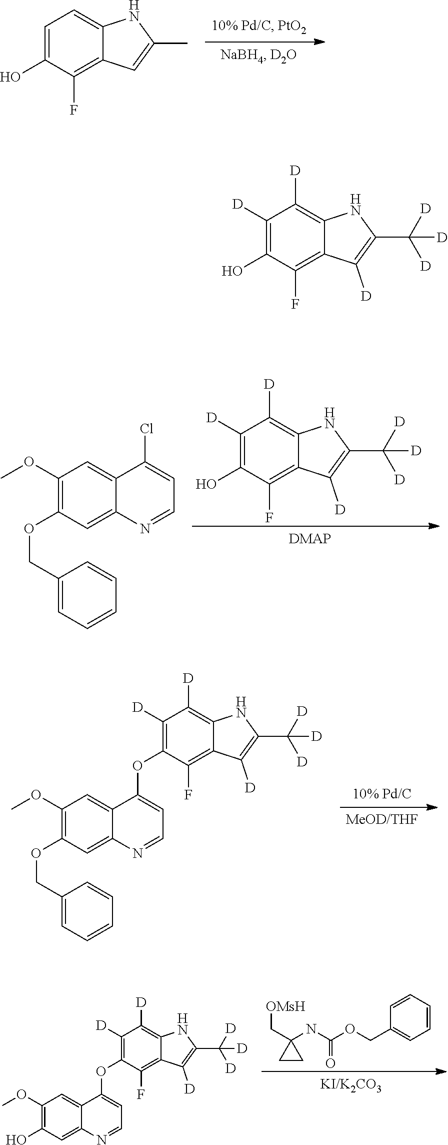

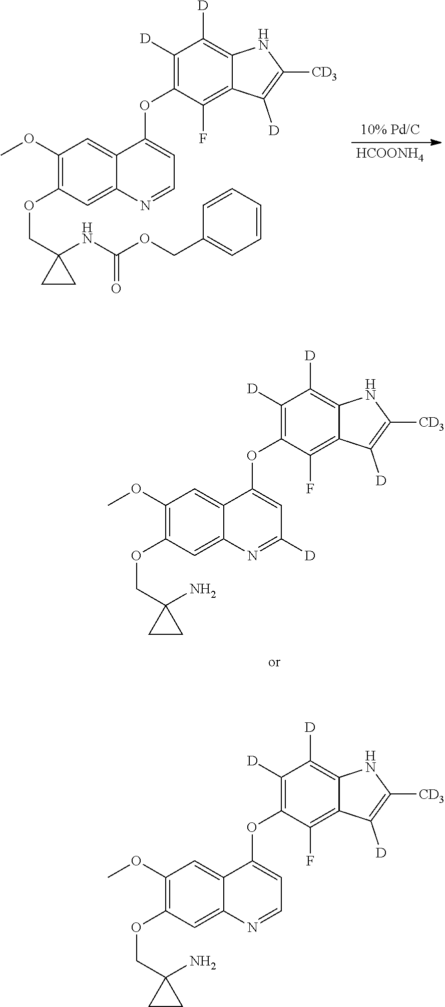 [SMILES: [2H]c1c(Oc2ccnc3cc(OCC4(NC(=O)OCc5ccccc5)CC4)c(OC)cc23)c(F)c2c([2H])c(C([2H])([2H])[2H])[nH]c2c1[2H] ; "HCOONH4" ; [2H]c1cc(Oc2c([2H])c([2H])c3[nH]c(C([2H])([2H])[2H])c([2H])c3c2F)c2cc(OC)c(OCC3(N)CC3)cc2n1 ; [2H]c1c(Oc2ccnc3cc(OCC4(N)CC4)c(OC)cc23)c(F)c2c([2H])c(C([2H])([2H])[2H])[nH]c2c1[2H]]

An example for preparing the deuterium substituted compound of Formula C is provided below. In some embodiments, step 1) is performed in the presence of CD₃I and NaH; in some embodiments, step 2) is performed in the presence of POCl₃.

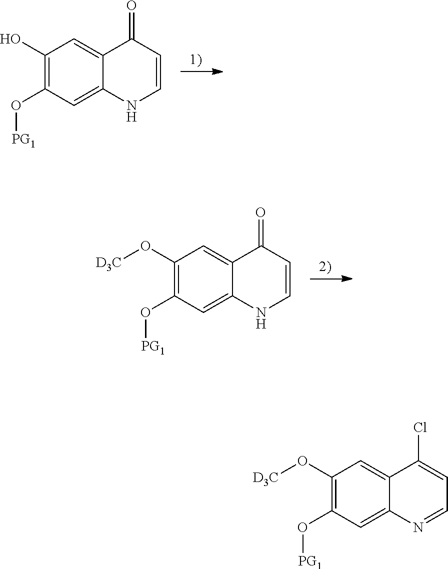

In some specific embodiments, the compound of Formula I-2 can be synthesized according to the following method.

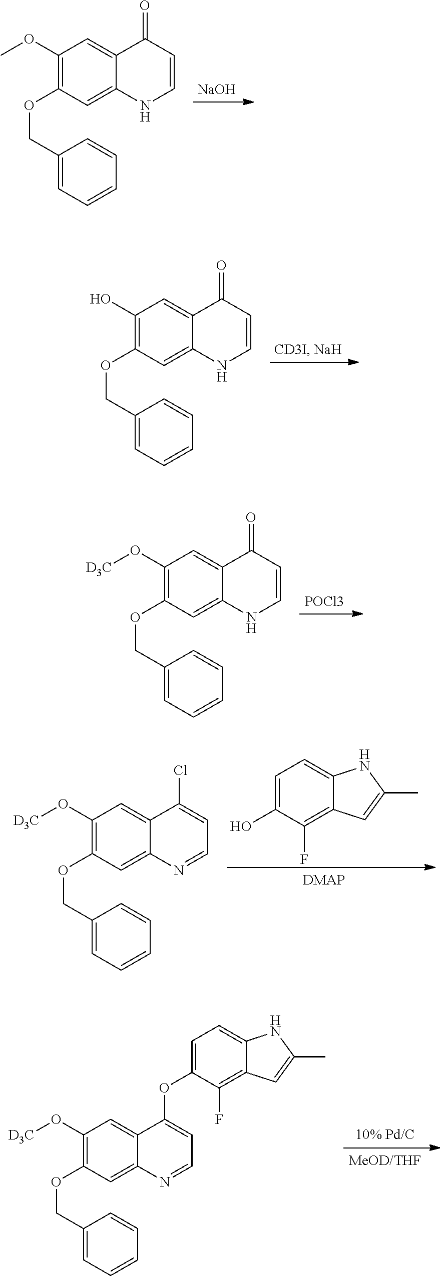

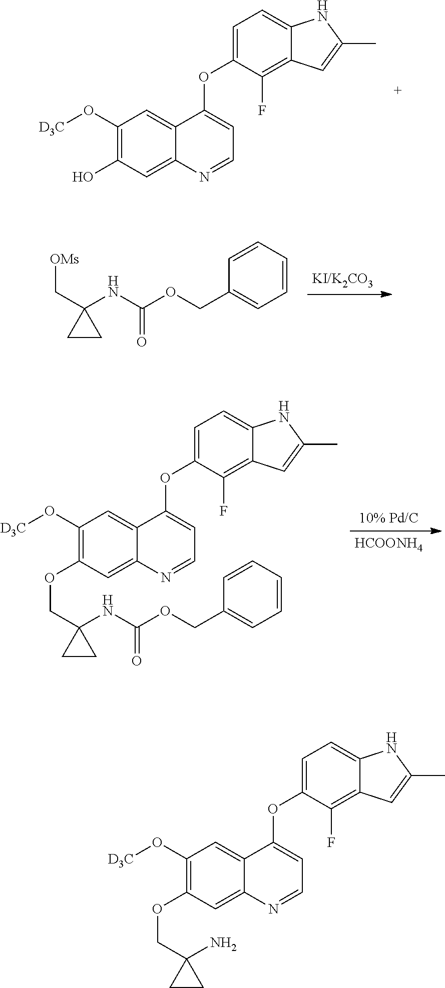 [SMILES: [2H]C([2H])([2H])Oc1cc2c(Oc3ccc4[nH]c(C)cc4c3F)ccnc2cc1O ; CS(=O)(=O)OCC1(NC(=O)OCc2ccccc2)CC1 ; [2H]C([2H])([2H])Oc1cc2c(Oc3ccc4[nH]c(C)cc4c3F)ccnc2cc1OCC1(NC(=O)OCc2ccccc2)CC1 ; "HCOONH4" ; [2H]C([2H])([2H])Oc1cc2c(Oc3ccc4[nH]c(C)cc4c3F)ccnc2cc1OCC1(N)CC1]

In some embodiments, an exemplary preparation method for the deuterium substituted compound of Formula F is provided as follows.

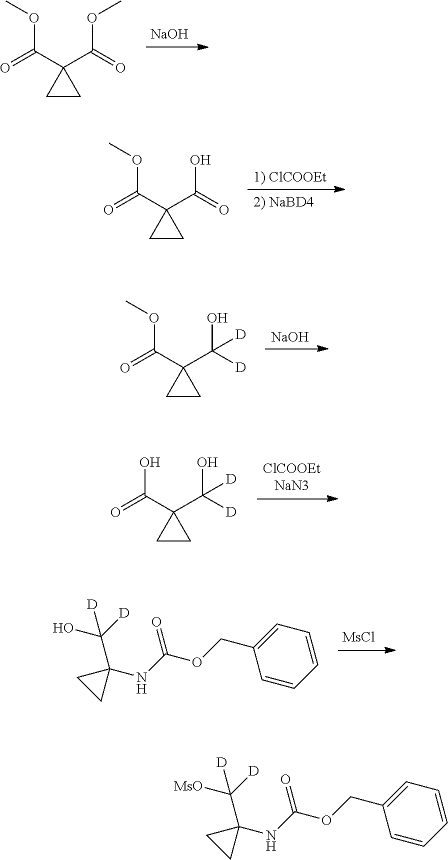

In some embodiments, a preparation method for the compound of Formula I-3 is provided as follows.

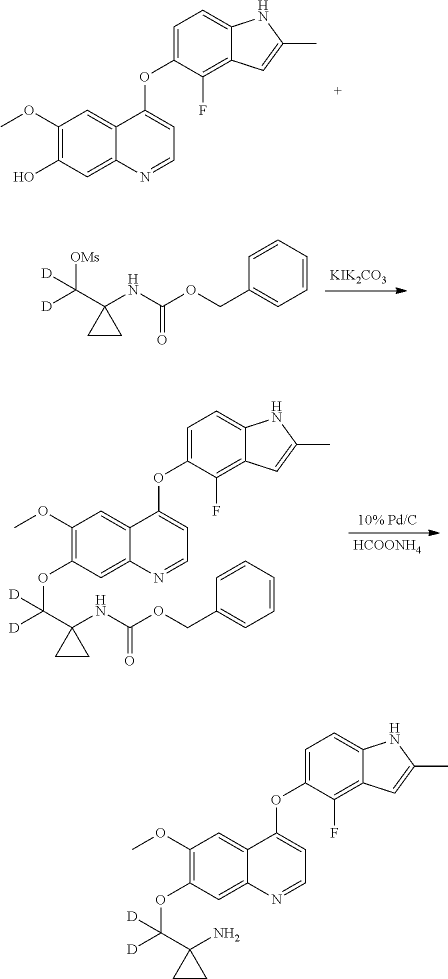

Definitions

Unless otherwise stated, the following terms used in this application have the following meanings. A particular term without a particular definition should be understood as having the ordinary meaning of the art. When a trade name appears in this document, it is intended to refer to its corresponding commodity or its active ingredient.

The term “H” means hydrogen.

The term “D” refers to deuterium.

The term “10% Pd/C” means 10% palladium on carbon.

The term “PtO₂” means platinum dioxide.

The term “D₂O” means deuteroxide.

The term “DCM” refers to dichloromethane.

The term “DMAP” means 4-dimethylaminopyridine.

The term “TLC” refers to thin layer chromatography.

The term “PE” refers to petroleum ether.

The term “EA” means ethyl acetate.

The term “1N HCl” means 1 mol/L of an aqueous solution of hydrochloric acid.

The term “K₂CO₃” means potassium carbonate.

The term “OMs” refers to methylsulfonyloxy.

The term “OTf” refers to trifluoromethylsulfonyloxy.

The term “OTs” refers to toluenesulfonyloxy.

The term “KI” refers to potassium iodide.

The term “THF” means tetrahydrofuran.

The term “DMF” is N,N-dimethylformamide.

The term “MeOH” refers to methanol.

The term “Ms” means methyl sulfonyl.

The term “HCOONH₄” is ammonium formate.

The term “HRMS” refers to a high resolution mass spectrum.

The term “substituted” means that any one or more hydrogens on the designated atom or ring is replaced with a selection from the indicated group, e.g. deuterium, provided that the designated atom's normal valency is not exceeded.

The term “hydrogen source” is a substance that reacts to produce hydrogen during the preparation process.

In this application, any atom not designated as deuterium exists with its natural isotopic abundance. When a particular position is deuterium, it can be understood that the abundance of deuterium at this position is substantially greater than the natural abundance of deuterium, and the natural abundance of deuterium is about 0.015%.

When any variable (e.g. R) occurs more than once in the composition or structure of a compound, its definition in each case is independent. Thus, for example, if a group is replaced by two R, then each R has an independent option.

The term “treating” means administering a compound or composition described herein to prevent, ameliorate or eliminate a disease or one or more symptoms associated with the disease, and includes:
(i) preventing a disease or disease state from occurring in a mammal, particularly when such a mammal is susceptible to the disease state but has not yet been diagnosed as having the disease state;
(ii) inhibiting the disease or disease state, i.e. curbing its development; or
(iii) alleviating the disease or disease state, even if the disease or disease state has subsided.

The term “therapeutically effective amount” means an amount of a compound of the present application effective for (i) treating or preventing a particular disease, condition or disorder, (ii) alleviating, ameliorating or eliminating one or more symptoms of a particular disease, condition or disorder, or (iii) preventing or delaying one or more symptoms of a particular disease, condition, or disorder described herein. The amount of a compound of the present application which constitutes a “therapeutically effective amount” will vary depending on the compound, the condition and severity thereof, the mode of administration, and the age of the mammal to be treated, but can be routinely determined by those skilled in the art based on their own knowledge and the content disclosed herein.

The term “pharmaceutically acceptable” which is for those compounds, materials, compositions and/or dosage forms is within the scope of sound medical judgment and is suitable for use in contact with human and animal tissues without too much toxicity, excitability, allergic reactions or other problems or complications, which is proportional to the ratio of reasonable benefit/risk.

As the pharmaceutically acceptable salt, for example, a metal salt, an ammonium salt, a salt formed with an organic base, a salt formed with an inorganic acid, a salt formed with an organic acid, a salt formed with a basic or acidic amino acid, or the like can be mentioned.

The term “pharmaceutical composition” refers to a mixture of one or more compounds of the present application or a salt thereof and a pharmaceutically acceptable adjuvant.

The term “pharmaceutically acceptable excipient” refers to those excipients which have no significant irritating effect on the organism and which do not impair the biological activity and properties of the active compound. Suitable excipients are well known to those skilled in the art, such as carbohydrates, waxes, water soluble and/or water swellable polymers, hydrophilic or hydrophobic materials, gelatin, oils, solvents, water, and the like.

The word “comprise” or “comprise” and its variants such as “comprises” or “comprising” shall be understood to mean an open, non-exclusive meaning, i.e. “including but not limited to”.

The pharmaceutical compositions of the present application can be prepared by combining the compounds of the present application with suitable pharmaceutically acceptable excipients, for example, as solid, semi-solid, liquid or gaseous preparations such as tablets, pills, capsules, powders, granules, ointments, emulsions, suspensions, suppositories, injections, inhalants, gels, microspheres and aerosols.

The pharmaceutical composition of the present application can be produced by a method well known in the art, such as a conventional mixing method, a dissolution method, a granulation method, a sugar coating pill method, a grinding method, an emulsification method, a freeze drying method, and the like.

In some embodiments, the pharmaceutical composition is in oral form. For oral administration, the pharmaceutical composition can be formulated by admixing the active compound with pharmaceutically acceptable excipients which are well known in the art. These excipients enable the compounds of the present application to be formulated into tablets, pills, troches, dragees, capsules, liquids, gels, slurries, suspensions and the like for oral administration to a patient.

Solid oral compositions can be prepared by conventional methods of mixing, filling or tableting. For example, it can be obtained by mixing the active compound with a solid adjuvant, optionally milling the resulting mixture, adding other suitable excipients if necessary, and then processing the mixture into granules to give tablets or the core of the dragee. Suitable excipients include, but are not limited to, binders, diluents, disintegrants, lubricants, glidants, sweeteners or flavoring agents, and the like.

The pharmaceutical compositions may also be suitable for parenteral administration, such as sterile solutions, suspensions or lyophilized products in a suitable unit dosage form.

Typical routes of administration of the compound of the present application, or a pharmaceutically acceptable salt thereof, or a pharmaceutical composition thereof include, but are not limited to, oral, rectal, topical, inhalation, parenteral, sublingual, intravaginal, intranasal, intraocular, intraperitoneal, intramuscular, subcutaneous, and intravenous administration.

### EXAMPLES

### Example 1

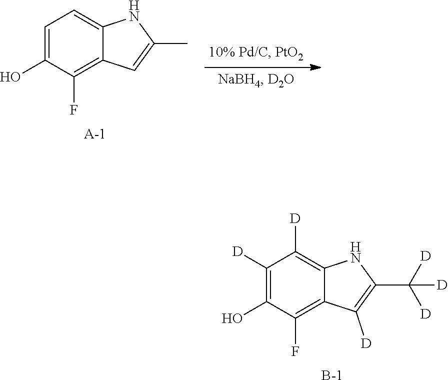

Compound A-1 (2.0 g), 10% Pd/C (0.20 g), PtO₂ (0.02 g), D₂O (20 ml) were added to a 35 ml microwave reaction flask, and mixed well at room temperature; NaBH₄ (0.01 g) was then added to the reaction mixture and stirred at room temperature for 5 minutes. The reaction was completed after six hours of microwave radiation (power 150W, 160° C.), the mixture was extracted with DCM (30 ml*3 times), and the organic phase was combined and dried over anhydrous sodium sulfate. The desiccant was removed by suction filtration, and the solvent was removed by rotary evaporation. The compound B-1 (1.36 g) was finally obtained by drying under reduced pressure at room temperature.

HRMS (ESI, [M+H]⁺) m/z: 172.1046.

### Example 2

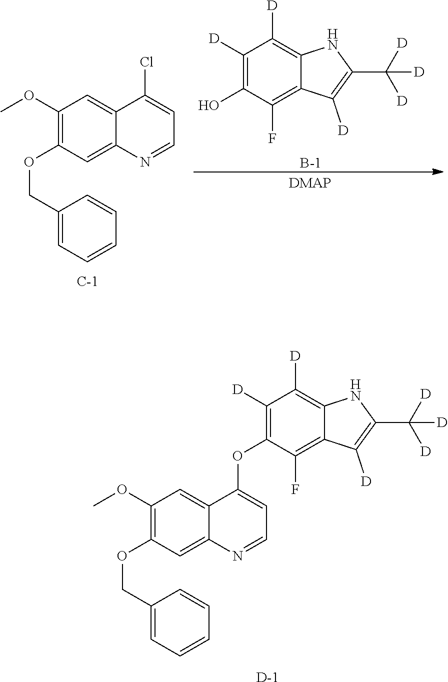

Compound C-1 (2.38 g), Compound B-1 (1.36 g), DMAP (1.94 g), 2,6-lutidine (12 ml) were added to a 250 ml round bottom single-neck flask, and the reaction mixture was heated to 140° C. After 4 hours, the reaction was completed after monitoring by TLC (developing solvent PE:EA=1:1). The mixture was cooled to room temperature and diluted with DCM (100 mL). 1N HCl (50 ml) was then added dropwise slowly, the organic phase was separated and washed with an aqueous solution of K₂CO₃, and then dried over anhydrous sodium sulfate. The desiccant was removed by suction filtration and the solvent was removed by rotary evaporation. The compound D-1 (2.25 g) was finally obtained by drying under reduced pressure.

HRMS (ESI, [M+H]⁺) m/z: 435.1828.

### Example 3

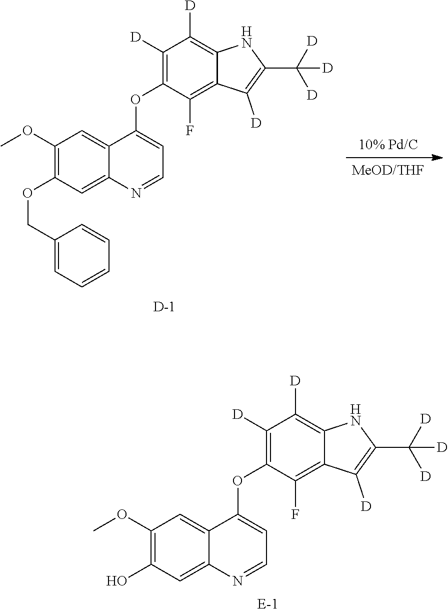

Compound D-1 (2.25 g), 10% Pd/C (0.50 g), MeOD (20 ml), THF (10 ml) were added to a 250 ml round bottom single-mouth flask and the system was washed three times with nitrogen, and then washed three times with hydrogen. The mixture was stirred at room temperature for 5 hours under 1 atmosphere of hydrogen. The reaction was completed after monitored by TLC (developing solvent DCM: MeOH=10:1). The desiccant was removed by suction filtration, and the solvent was removed by rotary evaporation. The compound E-1 (1.49 g) was finally obtained by drying under reduced pressure.

HRMS (ESI, [M+H]⁺) m/z: 345.1543.

### Example 4

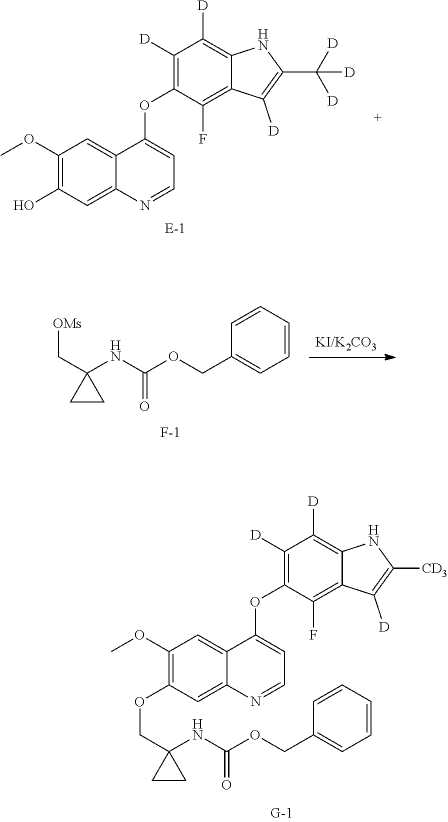

Compound E-1 (1.49 g), KI (2.52 g), K₂CO₃ (2.10 g), 2-butanone (40 ml) were added to a 250 ml round bottom flask, and the reaction mixture was heated to 60° C. Compound F-1 (1.30 g, added in four portions, added every 2 hours) was added in multiple portions. After reacting overnight, the reaction was completed after monitoring by TLC (developing solvent DCM:MeOH=10:1). The mixture was diluted with water and DCM; the mixture was separated and the aqueous phase was extracted with DCM (30 ml*3 times). The organic phase was combined, and dried over sodium sulfate. The desiccant was removed by suction filtration and the solvent was removed by rotary evaporation. The residue was separated by silica gel column chromatography (gradient elution: 100% DCM˜10% DCM:MeOH). Finally, the compound G-1 (2.36 g) was obtained by drying under reduced pressure.

HRMS (ESI, [M+H]⁺) m/z: 548.2205.

### Example 5

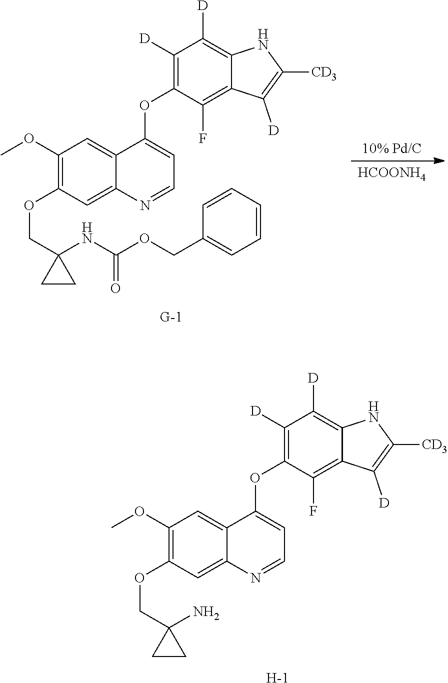

Compound G-1 (2.35 g), 10% Pd/C (1.0 g), HCOONH₄ (1.50 g), MeOD (35 ml) were added to a 250 ml round bottom flask, and the reaction mixture was heated to 50° C. The reaction was monitored by TLC (developer DCM:MeOD=10:1) and was complete after 1 hour. The catalyst was removed by suction filtration and the solvent was removed from the filtrate by rotary evaporation. The residue was separated through a silica gel column (gradient elution: 100% DCM˜5% DCM:MeOH). The product was combined, and the solvent was evaporated. Finally, the compound H-1 (0.864 g) was obtained by drying under reduced pressure.

¹H NMR (500 MHz, DMSO-d₆): δ11.45 (s, 1H), 7.616(s, 1H), 7.39(s, 1H), 6.33 (s, 1H), 4.11 (s, 2H), 3.99(s, 3H), 0.73 (dd, 4H).

What is claimed is:

1. A compound of Formula (I), or a pharmaceutically acceptable salt thereof, 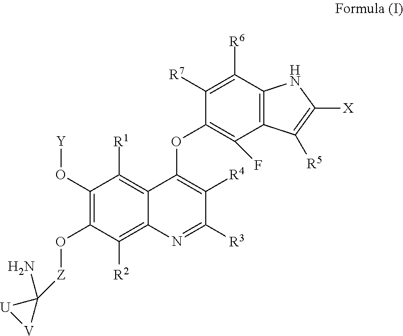
wherein each of X, and Y is independently C(R⁸)₃;
each of Z, U, and V is independently C(R⁹)₂; and
each of R¹, R², R³, R⁴, R⁵, R⁶, R⁷, R⁸, and R⁹ is independently hydrogen or deuterium;
provided that at least one of R¹, R², R³, R⁴, R⁵, R⁶, R⁷, R⁸, and R⁹ is deuterium.

2. The compound of claim 1, wherein at least one of R⁵, R⁶, R⁷, and R⁸ is deuterium.

3. The compound of claim 1, wherein at least one of R⁵, R⁶, and R⁷ is deuterium, and X is CD₃.

4. The compound of claim 1, wherein R⁵, R⁶, and R⁷ are deuterium, and X is CD₃.

5. The compound of claim 1, wherein Y is CD₃.

6. The compound of claim 1, wherein at least one of R¹, R², R³, and R⁴ is deuterium.

7. The compound of claim 1, wherein R³ is deuterium, R¹, R², and R⁴ are hydrogen.

8. The compound of claim 1, wherein at least one of R⁸, and R⁹ is deuterium.

9. The compound of claim 1, wherein at least one of Z, U, and V is CD₂.

10. The compound of claim 1, wherein Z is CD₂.

11. The compound of claim 1, wherein Z is CD₂, and U and V are CH₂.

12. The compound of claim 1, wherein Z is CH₂, and U and V are CD₂.

13. The compound of claim 1, wherein Z, U, and V are CD₂.

14. The compound of claim 1, wherein the compound is selected from the group consisting of: 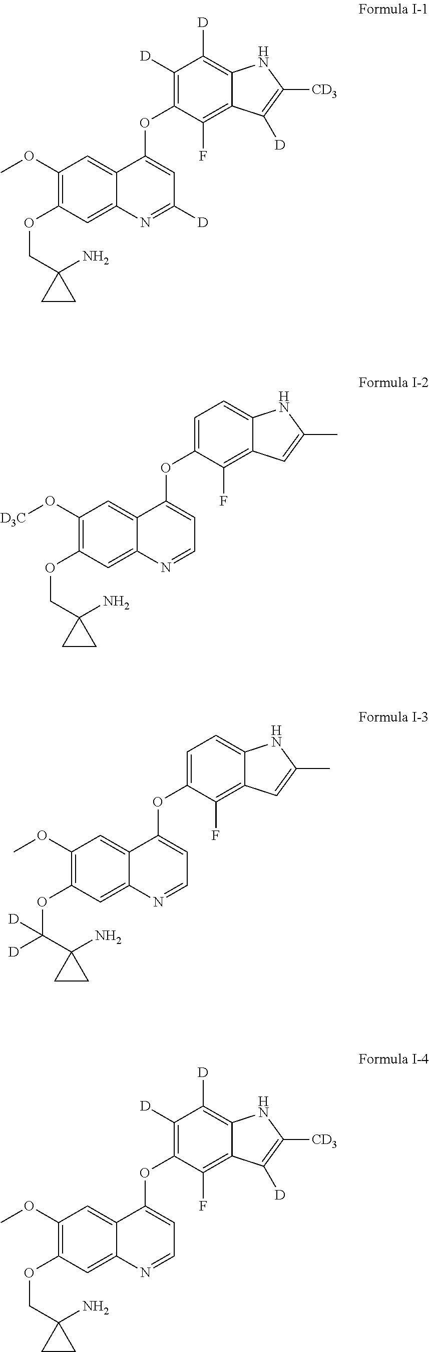 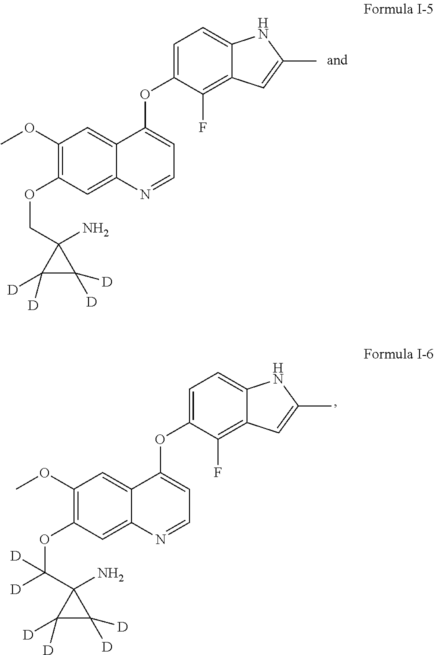
or pharmaceutically acceptable salts thereof.

15. A method for determining the concentration of anlotinib in a sample, comprising using a compound of claim 1 as an internal standard.

16. A pharmaceutical composition comprising a compound of claim 1 and a pharmaceutically acceptable excipient.

17. A process for the preparation of a compound of claim 1, comprising:
(1) reacting a compound of Formula C and a compound of Formula B in the presence of a base and a solvent to give a compound of Formula D;
(2) deprotecting the compound of Formula D to give a compound of Formula E;
(3) reacting the compound of Formula E and a compound of Formula F in a solvent in the presence of a base to give a compound of Formula G;
(4) converting the compound of Formula G to a compound of Formula H; 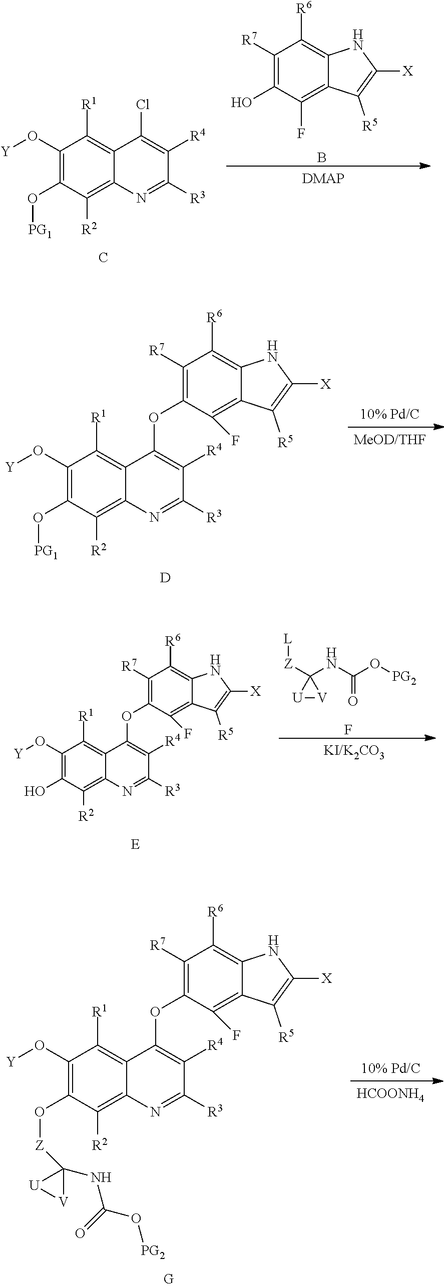 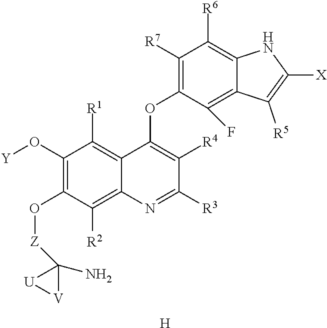
wherein each of X, and Y is independently C(R⁸)₃;
each of Z, U, and V is independently C(R⁹)₂; and
each of R¹, R², R³, R⁴, R⁵, R⁶, R⁷, R⁸, and R⁹ is independently hydrogen or deuterium;
PG₁ and PG₂ are protecting groups; and
L is a leaving group;
provided that at least one of R¹, R², R³, R⁴, R⁵, R⁶, R⁷, R⁸, and R⁹ is deuterium.

18. A process for preparing a compound of Formula B-1, comprising:
using the compound of Formula A-1 as a starting material.

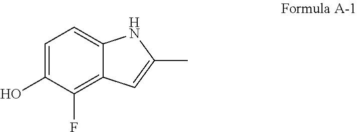

19. The process of claim 18, wherein the reaction is performed in the presence of a catalyst, D₂O, and H₂ or NaBH₄.

20. The process of claim 17, wherein
PG₁ and PG₂ are independently selected from the group consisting of benzyl, 2,4-dimethylbenzyl, 4-methoxybenzyl, 2,6-dichlorobenzyl, 3,4-dichlorobenzyl, and 4-(dimethylamino)carbonylbenzyl; and
L is selected from the group consisting of OMs, OTf, OTs, and Cl.
